(19) United States
(12) Patent Application Publication
(10) Pub. No.: US 2016/0332957 A1
(43) Pub. Date: Nov. 17, 2016

(54) PROCESS AND APPARATUS FOR PURIFYING A HYDROCARBON STREAM COMPRISING LINEAR ALPHA-OLEFINS (LAO) FROM AMINES

(71) Applicant: SAUDI BASIC INDUSTRIES CORPORATION, Riyadh (SA)

(72) Inventors: Syed Azhar Hashmi, Riyadh (SA); Mohammed H. Al-Hazmi, Riyadh (SA); Shahid Azam, Riyadh (SA)

(21) Appl. No.: 15/112,543

(22) PCT Filed: Jan. 19, 2015
(86) PCT No.: PCT/IB2015/050399

§ 371 Date: Jul. 19, 2016

Related U.S. Application Data
(60) Provisional application No. 61/929,138, filed on Jan. 20, 2014.

(51) Int. Cl. C07C 209/84; C07C 7/152; B01D 11/04; C07C 2/08

(52) U.S. Cl. CPC C07C 209/84; C07C 2/08; C07C 7/152; B01D 11/0492; B01D 11/0446

(57) ABSTRACT

A process for purifying linear a-olefins, comprises: providing an organic phase comprising a linear a-olefin; providing an alkaline aqueous phase I; providing an amine; mixing the linear a-olefin, alkaline aqueous phase I, and amine; separating the aqueous phase I from the organic phase; adding water to the organic phase thereby forming an aqueous phase II; bringing into contact the organic phase with carbon dioxide; separating the aqueous phase II which comprises a solid formed in the organic phase when contacted with carbon dioxide; wherein the purified linear a-olefins are obtained in the organic phase.

### TECHNICAL FIELD

The invention relates to a process for purifying a hydrocarbon stream comprising at least a linear alpha-olefin (a-olefin) (also referred to as: LAO) and at least an amine. The invention also relates to a process for manufacturing LAOs wherein at least one amine is separated from a hydrocarbon product stream comprising LAOs wherein an amine-carbon dioxide complex is obtained, and wherein the at least one amine and carbon dioxide are obtained from disintegrating the amine-carbon dioxide complex. The invention also relates to a process for separating one or more amines and carbon dioxide from at least one amine-carbon dioxide complex. The invention also relates to an apparatus in which the above-mentioned processes are carried out. The invention can be commercially utilized for the removal of one or more amines from one or more linear alpha-olefins (LAOs) or from a fraction of a product stream comprising one or more linear alpha-olefins (LAOs) by introducing carbon dioxide gas. The apparatus comprises a combination of decanters, heating vessel, injectors (for water and carbon dioxide) and piping system.

### BACKGROUND

In the chemical industry, processes are often conducted resulting in a product outlet stream or a feed stream to a process unit comprising hydrocarbons and amines. An example thereof is the outlet stream from a reactor utilized for preparing linear alpha-olefins (LAO) by oligomerization of ethylene. The linear alpha-olefins produced are separated into different fractions for further use or marketing. Often, one or more amines are added during the oligomerization process or into the reactor outlet piping system. In other processes, amines are utilized as corrosion inhibitors or for adjustment of the pH.

Often it is difficult to remove the amine from the hydrocarbon stream or fractions thereof by distillation since the boiling point of the amine and the LAO product are very close. Hence, it is considered to be too difficult and too expensive to separate the amines from the hydrocarbon stream by distillation. Further, it is described in the prior art that a simple conventional distillation is of no use for separating mixtures of components which have close boiling points. Moreover, azeotropic or extractive distillation cannot be used for separating the amine from the LAO product, since no adequate azeotrope forming agent or extraction agent has been identified so far.

Thus, there is an ongoing demand for a commercially available method to remove one or more amines from a hydrocarbon stream, or from a fraction thereof. The relatively high concentration of amine in the hydrocarbon stream is a further challenge.

European Patent Application No. 09 006 159.9 discloses a method for removing an organic amine from a hydrocarbon stream wherein the amine is reacted with an acid wherein a salt is obtained. The salt can be extracted into an aqueous phase. However, this method requires considerable investment cost because of the need for acid-resistant reactors and pipes.

### BRIEF SUMMARY

The present invention is generally based on the object of overcoming at least one of the problems encountered in the state of the art in relation to the purification of hydrocarbon product streams by separating amines therefrom.

A process for purifying linear a-olefins, comprises: providing an organic phase comprising a linear a-olefin; providing an alkaline aqueous phase I; providing an amine; mixing the linear a-olefin, alkaline aqueous phase I, and amine; separating the aqueous phase I from the organic phase; adding water to the organic phase thereby forming an aqueous phase II; bringing into contact the organic phase with carbon dioxide; separating the aqueous phase II which comprises a solid formed in the organic phase when contacted with carbon dioxide; wherein the purified linear a-olefins are obtained in the organic phase.

A process for purifying amines and carbon dioxide from amine-carbon dioxide complexes, comprises: providing an aqueous phase II comprising at least one complex of an amine and carbon dioxide, and a gas phase; heating the aqueous phase II; disintegrating the complex into at least an amine and carbon dioxide, wherein the carbon dioxide passes from the aqueous phase II into gas phase; wherein an aqueous phase II′ is formed comprising more amine than carbon dioxide; and wherein the gas phase comprises more carbon dioxide than amine.

A process for preparing linear a-olefins, comprises: providing an organic phase comprising at least ethylene and a catalyst; oligomerizing the ethylene wherein an organic phase comprising a linear a-olefin is obtained forming a compound (a); providing an alkaline aqueous phase I forming a compound (b); providing an amine forming a compound (c); mixing the compounds (a), (b), and (c); separating the aqueous phase I from the organic phase; adding water to the organic phase thereby forming an aqueous phase II; bringing into contact the organic phase with carbon dioxide at a pressure of 2.0 to 3.0 MegaPascals; separating the aqueous phase II which comprises a solid formed in the organic phase when brought into contact with carbon dioxide at a pressure of 2.0 to 3.0 MegaPascals; wherein the purified linear a-olefins are obtained in the organic phase; heating the aqueous phase II to 80100 ° C. at a pressure of 2.0 to 3.0 MegaPascals; disintegrating the complex into at least an amine and carbon dioxide, wherein the carbon dioxide passes from the aqueous phase II into gas phase; wherein the aqueous phase II contains more amine than carbon dioxide, and wherein the gas phase comprises more carbon dioxide than amine.

An apparatus comprises: a reactor having at least one feed and at least one outlet, wherein the product outlet is in serial fluid connection with a mixer, wherein at least two feeds are placed downstream of reactor but upstream of mixer; a decanter downstream of mixer with outlets; a decanter downstream of decanter, with outlets; at least a feed downstream of decanter but upstream or directly attached to decanter; and a heating device in fluid connection with outlet, wherein the heating device has a gas outlet in fluid connection with an inlet of decanter, and wherein the heating device has a liquid outlet in fluid connection with a decanter, wherein the decanter is in fluid connection with outlet and an outlet wherein outlet is in fluid connection with a pump, and wherein the pump is in fluid connection with the feed.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 shows a schematic view of the first aspect of the invention.

FIG. 2 shows a schematic view of the second aspect of the invention.

FIG. 3 shows a schematic view of the third aspect of the invention.

FIG. 4 is a schematic view of the fourth aspect of the invention.

### DETAILED DESCRIPTION

The present invention is based on the object of providing a process for purifying a hydrocarbon stream comprising at least a linear a-olefin. Another object is to provide a process for purifying a hydrocarbon stream comprising at least a linear a-olefin which is more resource efficient than prior art processes. Another object is to provide a process for purifying a hydrocarbon stream which is less energy-consuming than known processes. Another object is to provide a simple and easy to use process for the regeneration, separation and recycling of amines from amine containing salts.

A contribution to achieving at least one of the above described objects is made by the subject matter of the category forming the claims of the present invention. A further contribution is made by the subject matter of the dependent claims of the present invention which represent specific embodiments of the present invention.

Moreover, the process of the invention can be run using recycled and re-used process agents, in particular amine and carbon dioxide which reduces waste for disposal.

Moreover, the process of the invention does not require use of high purity carbon dioxide. Further, amine consumption and carbon dioxide consumption in the process are low because of recycling.

Moreover, amines are separated from product stream without using distillation steps or extraction steps. That way, effort for generation of process heat can be reduced. As a further benefit from not using distillation or extraction steps, capital investment cost can be decreased.

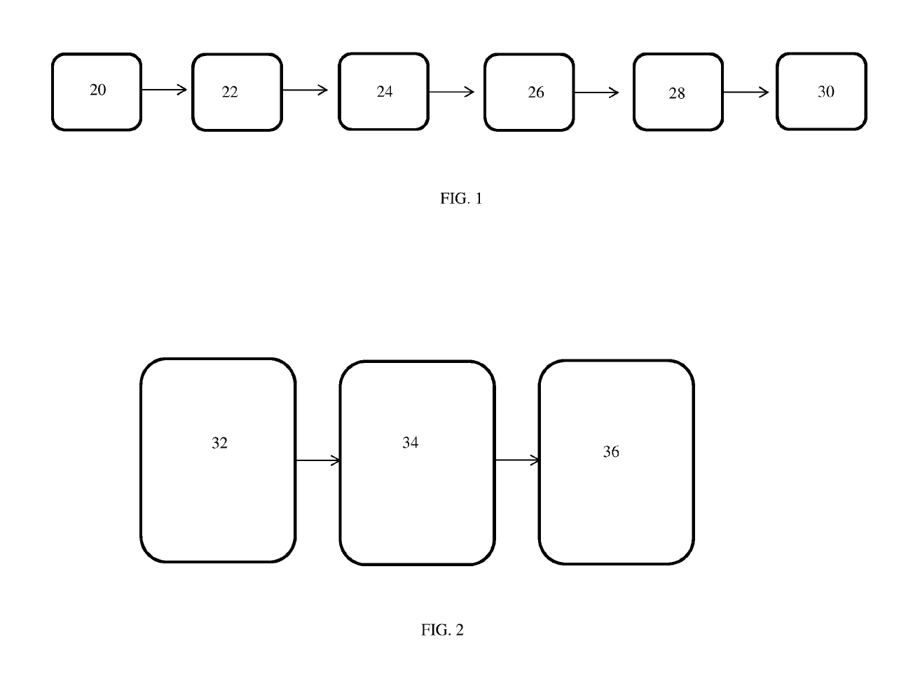

Turning now to FIG. 1, a first aspect of the invention which contributes to achieving at least one of the above-mentioned objects is a process for purifying linear a-olefins (LAOs) comprising providing an organic phase comprising a linear a-olefin 20; providing an alkaline aqueous phase I 20; providing an amine 20; mixing the linear a-olefin, alkaline aqueous phase I, and amine 22; separating the aqueous phase I from the organic phase 24; adding water to the organic phase thereby forming an aqueous phase II 26; bringing into contact the organic phase with carbon dioxide 28; separating the aqueous phase II which comprises a solid formed in the organic phase when contacted with carbon dioxide 30; wherein the purified linear a-olefins are obtained in the organic phase 30. For example, the aspect can be described as:

a. providing an organic phase comprising a linear a-olefin (LAO) 20;

b. providing an alkaline aqueous phase I 20;

c. providing an amine 20;

d. mixing a., b. and c 22;

e. separating the aqueous phase I from the organic phase 24;

f. adding water to the organic phase thereby forming an aqueous phase II 26;

g. bringing into contact the organic phase with carbon dioxide 28;

h. separating the aqueous phase II which comprises a solid formed in the organic phase in step g. 30;

wherein the purified linear a-olefins are obtained in the organic phase 30.

The organic phase of step a. comprises one or more linear a-olefins. A common acronym for a linear a-olefin is “LAO”. Plural: “LAOs”. LAOs can be obtained from ethylene oligomerization. The oligomerization of ethylene, catalysts for this reaction and suitable process conditions are known in the art. Preferred LAOs which can be subjected to the claimed process are C₄-C₃₀ fractions, preferably C₈-C₁₈ fractions, and more preferably 1-Octadiene, 1-Decadiene, 1-Dodecadiene, 1-tetradecene, 1-Hexadecadiene, 1-Octodecadiene, isomers of one or more of the aforementioned and combinations of two or more of any of the afore-mentioned LAO.

Preferably, the organic phase comprises 30 to 60 percent weight (% wt.), preferably 40 to 50% wt. of LAO, each time the % wt. with respect to the total weight of the organic phase.

Preferably, the organic phase comprises at least one further component which is non-polar. The term non-polar is understood to refer to a component, e.g. an organic solvent, which has no significant partial charges on any atoms, or where the polar bonds are arranged in such a way that the effects of their partial charges cancel out. Suitable examples of non-polar organic solvents are aliphatic or aromatic, saturated or unsaturated, linear, branched or cyclic hydrocarbons. Preferred organic solvents are aliphatic and aromatic hydrocarbons, preferably having 4 to 10 carbon atoms. More preferred organic solvents have 6 to 9 carbon atoms; still more preferred are benzene, toluene, ortho-, meta- and para-xylene, 1,3,5-trimethylbenzene, and heptane, or a combination of two or more of any of the aforementioned organic solvents. Most preferred is toluene or cyclohexane.

Preferably, the organic phase comprises 40 to 70% wt., preferably 50 to 60% wt. of the at least one further component, where the % wt. is measured with respect to the total weight of the organic phase.

In step b., an alkaline aqueous phase I is provided. There are numerous ways to prepare and thus numerous alkaline aqueous phases enter into consideration of those skilled in the art. Preferably, the alkaline aqueous phase I is selected from the group consisting of caustic potash solution, lime water, ammonium solution and caustic solution, or a combination of at least two thereof. Preferred is caustic solution.

Preferred concentrations of alkali in the alkaline aqueous phase I are 5 to 30% wt. of alkali, or 5 to 20% wt. of alkali, or 5 to 15% wt. of alkali. In a preferred embodiment, the aforementioned desired concentration can be obtained by mixing recycle alkaline aqueous solution, e.g. of a prior run of a step b. or the like, and fresh alkaline solution. The fresh alkaline solution preferably has a concentration of 10-30% wt. of alkali, or e.g. 20% wt. of alkali. A preferred alkali solution is caustic solution. Caustic solution is prepared by dissolving caustic soda (NaOH) in water. In a preferred embodiment, the volume ratio of aqueous phase to organic phase is 5:3 in step b.

In step c, at least one amine is provided. Numerous of the known amines enter into consideration of those skilled in the art. Preferably, the at least one amine is an organic amine selected from the group consisting of a primary, secondary, tertiary or cyclic amine, or a combination of at least two amines of same or different kind. Yet more preferred, the amine is selected from the group consisting of t-butyl amine, triethyl amine, cyclopentyl amine, t-octyl amine, n-heptyl amine, 2-heptyl amine, hexyl amine, 2-ethylhexyl amine, dihexyl amine, 1,6-diamino hexane, tributyl amine, 1,8-diamino octane, n-dodecyl amine, 3-ethylheptyl amine and tris-2-ethyl hexyl amine, or a combination of at least two thereof.

Preferably, amines are added as pure substances, or in technical grade. Technical grade reagents often comprise some amounts of impurities. Further preferred embodiments comprise adding amines in form of aqueous or organic solution. In organic solution, the amine is preferably present in an amount of 0.1 to 5% wt., or 0.1 to 2% wt., or 0.2 to 0.5% wt., based on the total amount of the organic solution.

In step d. the organic phase comprising LAO, the alkaline aqueous phase I and the at least one amine are mixed. Numerous ways to mix multiple components as in step d. enter into consideration of those skilled in the art. Preferably, in step d. mixing is performed using a dynamic mixer or a static mixer. A particularly preferred method of mixing is using a static mixer. Numerous designs of static mixers are known to those skilled in the art. Preferred designs of static mixers comprise a tubular housing and a series of baffles. Both housing and baffles are preferably made of a material which is not deteriorated by contact with the organic phase or with the aqueous phase I. In another preferred embodiment, housing and baffles can be made of a material which is sensitive to contact with the organic phase or with the aqueous phase I. In this event, the surface of those parts of the mixer is covered with a tight layer of protective material which are in contact with the organic phase or with the aqueous phase I. Then, the protective material is inert, or at least resistant, to the organic phase or with the aqueous phase I.

In a further preferred embodiment of the process of the invention, at least a part of, preferably the complete step d. and/or e. is performed at a temperature of 60 to 90° C., more preferred 70 to 85° C.

In step e., at least a part of the aqueous phase I is separated from the organic phase. Preferably, in step e., more than 50% wt., or more than 70% by wt, or more than 20 90% wt., based on the total weight of the organic phase at the beginning of step e., are separated from the organic phase. Often, more than 95% wt., or more than 98% wt., based on the total weight of the organic phase at the beginning of step e., are separated from the organic phase in step e.

Numerous ways to separate an aqueous phase from an organic phase enter into consideration of those skilled in the art. Preferably, separating in step e. is performed using a decanter. Preferred decanters have a length-to-diameter ratio (in the following referred to as: L/D ratio) of 1 to 5, more preferred 1.3-3, or 1.5 to 2.5.

In step f., water is added to the organic phase wherein an aqueous phase II is formed. Numerous ways to add water to the organic phase are known to those skilled in the art through which is formed an aqueous phase II. Preferably, the line which conveys the organic phase is joined by a water line fitted with an injector or a mixer.

In step g., the organic phase and aqueous phase II are brought into contact with carbon dioxide. Numerous ways to bring the organic phase and the aqueous phase II into contact are known to those skilled in the art. Preferably a pressured line of carbon dioxide joins the line which conveys the organic phase. Any type of injector can be used at the joint. Preferably, the carbon dioxide has a pressure of 2.0 to 3.0 MegaPascals (MPa) (20 to 30 bar) when being joined to the organic phase.

Upon contact of amine and carbon dioxide, an amine-carbon dioxide complex is formed. The term “complex” is used to describe that amine and carbon-dioxide species interact to form agglomerates. However, the term “complex” is not used to describe any particular type of interaction between amine and carbon dioxide species. The term “complex” can comprise any liaison between both sorts of chemical compound, e.g. ionic, van-der-Vaals, complex, electrostatic and so on.

The amine-carbon dioxide complex comprises at least partially a complex salt of amine and carbon dioxide which preferably is less soluble in the organic phase than the amine. Preferably, at least some of the amine-carbon dioxide complex species is accepted by aqueous phase II.

In step h., aqueous phase II which comprises at least some of the amine-carbon dioxide complex species is separated from the organic phase. Preferably, in step h., more than 90% wt., or more than 95% wt., or more than 99% wt., based on the total weight of the aqueous phase II present at the beginning of step h., are separated from the organic phase.

Numerous ways to separate an aqueous phase from an organic phase enter into consideration of those skilled in the art. Preferably, separating in step h. is performed using a decanter. Preferred decanters have a length-to-diameter ratio (in the following referred to as: L/D ratio) of 1 to 5, more preferred 2.3-4, or 2.5 to 3.5.

In another preferred embodiment, steps g. and h. are both performed using a decanter. In this embodiment, the decanter has an inlet for the line conveying both the organic phase and the aqueous phase II, and another inlet for a carbon dioxide feed line upstream, prior to the decanting section of the decanter. The L/D ratio of the decanter and further embodiments are the same as has been described above for the decanter, in which step h. alone can be applied.

After separating of the aqueous phase in step h., purified linear a-olefins (pl_AO) are obtained. The purified LAO can be stored or used for further treatment or chemical conversion reactions downstream.

Preferably, the pLAO comprises less than 100 parts per million (ppm) of amine. According to another preferred embodiment, pLAO contains less than 10 ppm of amine.

In a preferred embodiment of the process of the invention, at least step g. is performed at a pressure of more than 2.0 MPa (20 bar), preferably 2.1 MPa to 5.0 MPa (21 bar to 50 bar), or 2.1 to 40 MPa (21 to 40 bar), or 2.0 to 3.0 MPa (20 to 30 bar).

In a preferred embodiment of the process of the invention, the process is performed as a continuous process. The term “continuous” is used in the context of this invention when all steps of the process are operated in a defined way without manual interaction by a human. It is often seen as opposite to “batch” operations.

In a preferred embodiment of the process of the invention, the linear a-olefin present in the organic phase is obtained by oligomerizing ethylene. Oligomerization is any chemical process that converts monomers to macromolecular complexes through a finite degree of polymerization. Oligomerizing describes the action of conducting such process. Accordingly oligomerization of ethylene describes any chemical process that converts 2 to 10 equivalents of ethylene into one coherent molecular structure of 2 to 10 repeat derived from ethylene.

In a preferred embodiment of the process of the invention, no distillation step is used to separate the amine from the organic phase.

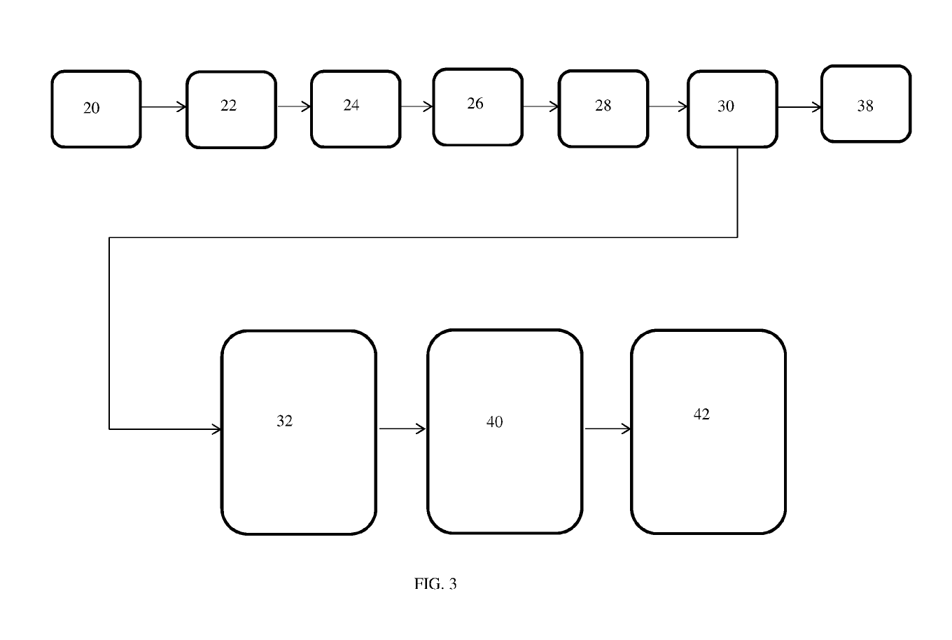

FIG. 2 illustrates a second aspect of the invention which contributes to achieving at least one of the above-mentioned objects is a process for purifying amines and carbon dioxide from amine-carbon dioxide complexes comprising

a) providing an aqueous phase II comprising at least one complex of an amine and carbon dioxide, and a gas phase 32;

b) heating the aqueous phase II 34;

c) disintegrating the complex into at least an amine and carbon dioxide 36,

wherein the carbon dioxide passes from the aqueous phase II into gas phase;

wherein an aqueous phase II′ is formed comprising more amine than carbon dioxide, and

wherein the gas phase comprises more carbon dioxide than amine.

In step a), an aqueous phase II is provided which comprises at least a complex of an amine and carbon dioxide, and a gas phase. In a preferred embodiment, the aqueous phase II is a product of the process according to the first aspect of the invention which is described above. Preferably, aqueous phase II has in at least one moment during step a) a temperature of 70 to 90° C., or 75 to 85° C.

In step b), aqueous phase II is heated in a heater. Preferably, aqueous phase II is heated to a temperature of 60 to 90° C., or 70 to 85° C. Numerous heaters are known and considered by to those skilled in the art. Preferably, aqueous phase II is heated by guiding it through a tubular heat exchanger.

In a preferred embodiment of the process of the invention, at least step b) is performed at a pressure of more than 2.0 MPa (20 bar), preferably 2.1 MPa to 4.0 MPa (21 bar to 50 bar), or 2.1 to 4.0 MPa (21 to 40 bar), or 2.0 to 3.0 MPa (20 to 30 bar).

In step c), which occurs at elevated temperature (compared to the temperature in step a)), the complex disintegrates into at least an amine and carbon dioxide. Disintegration is achieved substantially completely. The carbon dioxide migrates from aqueous phase II into the gas phase, which is located on top of the heated aqueous phase. Thereby amine enriched aqueous phase if is formed. The aqueous phase if then comprises more amine than carbon dioxide. The amine enriched aqueous phase II′ preferably comprises less than 100 ppm, or less than 10 ppm of amine, with respect to the total aqueous phase II′. The gas phase comprises more carbon dioxide than amine.

Preferably, the gas phase is removed on top of the heater. One option is to deliberate the carbon dioxide into environment. Preferred is to recycle the carbon dioxide to the carbon dioxide feed of another process, e.g. to the process step g. of the first contribution to the present invention. Further, the heater comprises at least an outlet to remove aqueous phase II′ which is amine enriched.

In an embodiment of the second aspect of the invention, at least some water is removed from the aqueous phase II′, wherein an amine enriched (aqueous) phase II″ is obtained.

In an embodiment of the second aspect of the invention, the process is a continuous process.

FIG. 3 illustrates a third aspect of the invention which contributes to achieving at least one of the above-mentioned objects is a process for preparing linear a-olefins (LAOs) comprising

(1) providing an organic phase comprising at least ethylene and a catalyst;

(2) oligomerizing the ethylene wherein a an organic phase comprising a linear a-olefin (LAO) is obtained 20;

(3) providing an alkaline aqueous phase I 20;

(4) providing an amine 20;

(5) mixing the compounds of step (2), (3) and (4) 22;

(6) separating the aqueous phase I from the organic phase 24;

(7) adding water to the organic phase thereby forming an aqueous phase II 26;

(8) bringing into contact the organic phase with carbon dioxide 28;

(9) separating the aqueous phase II which comprises a solid formed thereby formed in the organic phase in step (8) 30;

wherein the purified linear a-olefins are obtained in the organic phase 38 and wherein the aqueous phase II comprising amine-carbon dioxide complex and gas phase is provided;

(10) heating the aqueous phase II 40;

(11) disintegrating the complex into at least an amine and carbon dioxide, wherein the carbon dioxide passes from the aqueous phase II into gas phase 40;

wherein an aqueous phase II is formed comprising more amine than carbon dioxide 42, and

wherein the gas phase comprises more carbon dioxide than amine 42.

The third aspect of the invention essentially combines the process of the first aspect of the invention and the process of the second aspect of the invention, wherein steps (3) to (9) correspond to steps b. to h. of the first aspect of the invention. Steps (10) and (11) correspond to step b) and c) of the second aspect of the invention. Preferred features and embodiments of the third aspect of the invention correspond to the features and embodiments of the first and second aspect of the invention and are incorporated by reference herewith.

In general, steps (1) and (2) can be performed using a process known in the art. For example, such a process is described in EP Patent Publication No. 2 287 142 A1. EP Patent Publication No. 2 287 142 A1 is incorporated herein by reference in full, in particular the method of manufacture of LAO.

In an embodiment of the third aspect of the invention, at least some water from the aqueous phase II′ is removed in a further step (12), wherein an amine-enriched, preferably aqueous phase II″ is obtained.

In an embodiment of the third aspect of the invention, the process is a continuous process. Preferably, aqueous phase if or aqueous II″ which are obtained in step (11) or in step (12) is combined with the amine from a make-up line prior to being provided for mixing in step (4). Preferably, the gas phase obtained in step (11) is combined with a carbon dioxide feed from a make-up line for providing carbon dioxide in step (8).

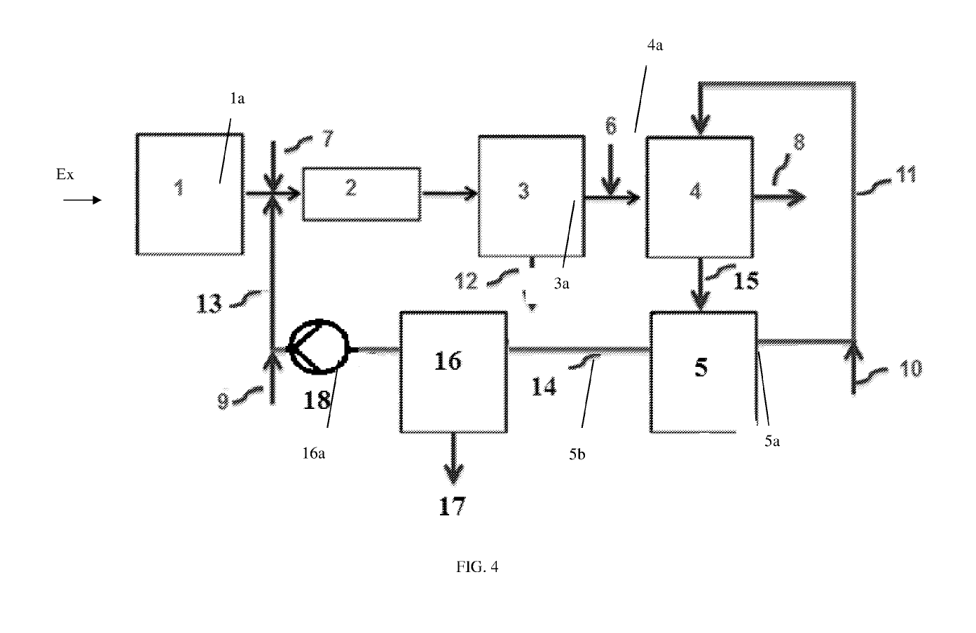

Turning now to FIG. 4, a fourth aspect of the invention which contributes to achieving at least one of the above-mentioned objects is an apparatus comprising

A) a reactor 1 which has at least one feed Ex and at least one outlet 1a, wherein

B) the reactor 1 is in serial fluid connection with a mixer 2, wherein

C) at least two feeds 7, 13 are placed downstream of reactor 1 but upstream of mixer 2, and

D) a decanter 3 downstream of mixer 2, with outlets 12 and 3a,

E) a decanter 4 downstream of decanter 3, with outlets 15 and 8 and

F) at least a feed 6 downstream of decanter 3 but upstream or directly attached to decanter 4, and

G) a heating device 5 in fluid connection with outlet 15,

wherein the heating device 5 has a gas outlet 5a, preferably in the to part of heating device 5, which is in fluid connection, optionally via a pump, with an inlet 4a of decanter 4, or in alternative with feed 6, and

wherein the heating device 5 has a liquid outlet 5b, preferably in the bottom part of heating device 5, in fluid connection with a decanter 16, wherein

H) the decanter 16 is in fluid connection with outlet 17, preferably in the bottom part of decanter 16, and an outlet 16a, preferably in the top part of decanter 16, wherein outlet 16a is in fluid connection with pump 18, and wherein

I) the pump 18 is in fluid connection with feed 13.

Numerous types of reactors enter into consideration of those skilled in the art. Preferably, the reactor is a column reactor, e.g. a bubble column reactor. Preferably, the reactor has a length to diameter (L/D) ratio of 1 to 10, or 2 to 5, or 2 to 3. Preferably, the reactor has one or more feeds, e.g., Ex, wherein each feed, Ex, can convey one or more educts for the reaction to be carried out in the reactor. A preferred reaction is an olefin polymerization or an olefin oligomerization, preferably of ethylene or propylene. As well, a feed Ex can be used to feed catalyst into the reactor.

Numerous types of mixers enter into consideration of those skilled in the art. Examples of suitable mixers are described in the above regarding step d. of the first aspect of the invention. These mixers and mixing devices are incorporated herein. Preferred embodiments described there are also incorporated herein.

Numerous types of decanters useful as decanter 3 enter into consideration of those skilled in the art. Examples of suitable decanters as decanter 3 are described in the above regarding step e. of the first aspect of the invention. These decanters are incorporated herein. Preferred embodiments described there are also incorporated herein. Outlet 12 is preferably used for conveying an aqueous phase. Outlet 3a is preferably used for conveying the hydrocarbon stream comprising LAO.

Numerous types of decanters useful as decanter 4 enter into consideration of those skilled in the art. Examples of suitable decanters as decanter 4 are described in the above regarding step h. of the first aspect of the invention. These decanters are incorporated herein. Preferred embodiments described there are also incorporated 15 herein. Outlet 4b is preferably used for conveying an aqueous phase. Outlet 4c is preferably used for conveying the hydrocarbon stream comprising LAO, now purified.

Numerous types of heating devices useful as heating device 5 enter into consideration of those skilled in the art. Examples of heating devices suitable for use as heating device 5 are a heating reactor, a heating vessel, a heat exchanger, preferably a plate heat exchanger, a concentric tube heat exchanger, a plate fin heat exchanger and a shell and tube heat exchanger, or a combination of two or more of the aforementioned heat exchanger, of same or different type.

Numerous types of decanters useful as decanter 16 enter into consideration of those skilled in the art. Examples of suitable decanters as decanter 16 are described in the above regarding step h. of the first aspect of the invention. These decanters are incorporated herein. Preferred embodiments described there are also incorporated herein. Outlet 17 is preferably used for conveying an aqueous phase. Outlet 16a is preferably used for conveying the amine which is now recycled. Numerous types of pumps useful as item I) enter into consideration of those skilled in the art.

Various pieces of pipe are used to connect the various items of the apparatus. The hydrocarbon product line 8 preferably conveys an organic phase comprising at least one LAO. The hydrocarbon product line 8 is connected upstream to outlet la of reactor 1 and then downstream to mixer 2, further connecting an outlet of mixer 2 downstream with inlet of decanter 3, further connecting outlet 3a of decanter 3 with an inlet of decanter 4, further conveying the organic phase comprising at least one LAO—now purified—from outlet 4c to storage vessels or further processing, or both.

The aqueous stream comprising aqueous phase with amine carbon dioxide complex in line 15 originates from outlet 4b to an inlet of heating device 5.

The amine/water stream transfer line 14 originates from outlet 5b to an inlet of decanter 16.

From outlet 16, there is a line 19 conveying amine—now purified—via pump 18 to feed 13.

Feeds are positioned at various positions of the piping and at some devices of the apparatus. Numerous types of feeds enter into consideration of those skilled in the art. The type and device of feed is chosen by the skilled person according to the fed compound and/or physical state thereof in line with his common general knowledge. Preferred feeds are liquid injectors, gas injectors, optionally with check-valves, sensor, regulators and so on. In a very simple fashion, a feed can be formed by a pipe which joins another pipe conveying a stream which is further conveyed downstream.

A fifth aspect of the invention is a process according the process of to the third aspect of the invention which is conducted in the apparatus of the fourth aspect of the invention. Preferred embodiments of both, the third aspect and the fourth aspect of the invention, are incorporated herein as embodiments to the fifth aspect of the invention.

FIG. 4 shows a greatly simplified embodiment of a system employing the inventive processes. The product stream coming out from the reactor 1 is passed into a mixer 2 where it is mixed with amine solution of concentration (0.3-0.8% wt.) coming from line 9 and caustic solution of concentration (15-30% wt.) coming from line 7. The product stream coming out from mixer 2 is conveyed into decanter 3 where an aqueous layer is separated and removed from the product stream through outlet 12. The organic product stream coming out of decanter 3 is then passed into decanter 4 which has a water injection line 6, and a carbon dioxide injection line 13. The product stream leaves decanter 4 through line 8. The water stream containing an amine-carbon dioxide complex is continuously withdrawn via outlet 4b and sent into the heating device 5 through line 15. In the heating device 5 the amine-carbon dioxide complex gets disintegrates at temperature in of about 80 to 100° C., and at a pressure ranging of about 2.1 MegaPascals (MPa) to about 3.0 MPa (about 21 bar to about 30 bar). The carbon dioxide gas exits heating vessel 5 through gas outlet 5a via carbon dioxide recycle line 11 to the inlet 4a at decanter 4. Further, make-up carbon dioxide is injected from line 10 into line 11. The amine-water stream is then transferred from outlet 5b of heating vessel 5 through line 14 to decanter 16 where amine and water are separated. Amine is conveyed from outlet 16a of decanter 16 through line 19 back to feed 13, downstream of reactor 1 using pump 18. Further make-up amine is added through line 9 into line 19. The aqueous stream is removed from the system through outlet 17.

The following example is merely illustrative of the device disclosed herein and is not intended to limit the scope hereof.

### EXAMPLE

The example was performed using an apparatus as described in FIG. 4. The product stream (mainly comprising 1-octadiene) was obtained from a reactor and was passed into a static mixer (M). There, it was mixed with a feed of amine solution of concentration (0.3wt.%) which combined recycled amine solution and fresh amine (if necessary), and another feed with caustic solution of concentration (20 wt.%). The product stream which come out from the static mixer (M) was conveyed into a first decanter of L/D ratio=3, where an aqueous layer was separated and removed from the product stream through an outlet. The organic product stream which came out of the first decanter was then passed into a second decanter. Water was added and carbon dioxide was injected by a carbon dioxide injector into the liquid phase in the second decanter. A stream of purified product left the second decanter through a line for further processing. The aqueous phase which left the second decanter contained a solid (amine-carbon dioxide complex). The aqueous phase containing the solid was conveyed into a heating vessel where it was heated to a temperature of 85° C. at a pressure of 24.7 bar. There, the solid disintegrated and discharged carbon dioxide. The carbon dioxide gas was transferred through a line and conveyed to the carbon dioxide injector (CI) of the second decanter. Additional (fresh) carbon dioxide was added if necessary to maintain pressure and carbon dioxide concentration. The aqueous phase was transferred from the heating vessel to a third decanter where amine (=organic phase) and water were separated. The amine was transferred from the third decanter via a pump to static mixer (M). Fresh amine was added if necessary. The aqueous phase present in the third decanter was removed via an outlet. The aforementioned run delivered a purified stream of LAO while generation of waste was minimized. This has been realized by recycling and reusing both carbon dioxide and amine.

The process and apparatus disclosed herein includes at least the following embodiments:

Embodiment 1: A process for purifying linear a-olefins, comprising: providing an organic phase comprising a linear a-olefin; providing an alkaline aqueous phase I; providing an amine; mixing the linear a-olefin, alkaline aqueous phase I, and amine; separating the aqueous phase I from the organic phase; adding water to the organic phase thereby forming an aqueous phase II; bringing into contact the organic phase with carbon dioxide; separating the aqueous phase II which comprises a solid formed in the organic phase when contacted with carbon dioxide; wherein the purified linear a-olefins are obtained in the organic phase.

Embodiment 2: The process of claim 1, wherein at least the bringing into contact the organic phase with carbon dioxide is performed at a pressure of more than 2.0 MegaPascals.

Embodiment 3: The process of claim 1 or 2, wherein the process is performed as a continuous process.

Embodiment 4: The process of any of claims 1 to 3, wherein the amine is an organic amine selected from a primary, secondary, tertiary or cyclic amine.

Embodiment 5: The process of claim 4, wherein the organic amine is selected from t-butyl amine, triethyl amine, cyclopentyl amine, t-octyl amine, n-heptyl amine, 2-heptyl amine, hexyl amine, 2-ethylhexyl amine, dihexyl amine, 1,6-diamino hexane, tributyl amine, 1,8-diamino octane, n-dodecyl amine, 3-ethylheptyl amine and tris-2-ethyl hexyl amine.

Embodiment 6: The process of any of claims 1 to 5, wherein the organic phase comprises at least one component which is non-polar.

Embodiment 7: The process of claim 6, wherein the organic phases further comprises at least and organic solvent selected from aromatic and aliphatic solvents

Embodiment 8: The process of claim 7, wherein the organic solvent is toluene or cyclohexane.

Embodiment 9: The process of any one of claims 1 to 8, wherein the linear a-olefin is obtained by oligomerizing ethylene.

Embodiment 10: A process for purifying amines and carbon dioxide from amine-carbon dioxide complexes, comprising: providing an aqueous phase II comprising at least one complex of an amine and carbon dioxide, and a gas phase; heating the aqueous phase II; disintegrating the complex into at least an amine and carbon dioxide, wherein the carbon dioxide passes from the aqueous phase II into gas phase; wherein an aqueous phase if is formed comprising more amine than carbon dioxide; and wherein the gas phase comprises more carbon dioxide than amine.

Embodiment 11: The process of claim 10, wherein at least the heating of the aqueous phase II is performed at a pressure in of more than 2.0 MegaPascals.

Embodiment 12: The process of claim 10 or 11, wherein the heating of the aqueous phase II is performed until the temperature of the aqueous phase II is 80-100° C.

Embodiment 13: The process of any of claims 10 to 12, wherein at least some water is removed from the aqueous phase II′, wherein an amine enriched aqueous phase II″ is obtained.

Embodiment 14: The process of any of claims 10 to 13, wherein the process is a continuous process.

Embodiment 15: A process for preparing linear a-olefins, comprising: providing an organic phase comprising at least ethylene and a catalyst; oligomerizing the ethylene wherein an organic phase comprising a linear a-olefin is obtained forming a compound (a); providing an alkaline aqueous phase I forming a compound (b); providing an amine forming a compound (c); mixing the compounds (a), (b), and (c); separating the aqueous phase I from the organic phase; adding water to the organic phase thereby forming an aqueous phase II; bringing into contact the organic phase with carbon dioxide at a pressure of 2.0 to 3.0 MegaPascals; separating the aqueous phase II which comprises a solid formed in the organic phase when brought into contact with carbon dioxide at a pressure of 2.0 to 3.0 MegaPascals; wherein the purified linear a-olefins are obtained in the organic phase; heating the aqueous phase II to 80-100° C. at a pressure of 2.0 to 3.0 MegaPascals; disintegrating the complex into at least an amine and carbon dioxide, wherein the carbon dioxide passes from the aqueous phase II into gas phase; wherein the aqueous phase II contains more amine than carbon dioxide, and wherein the gas phase comprises more carbon dioxide than amine.

Embodiment 16: The process of claim 15, further comprising removing at least some water from the aqueous phase II, wherein an amine enriched phase II is obtained.

Embodiment 17: The process of any one of claim 15 or 16, wherein the process is a continuous process.

Embodiment 18: An apparatus, comprising: a reactor having at least one feed and at least one outlet, wherein the product outlet is in serial fluid connection with a mixer, wherein at least two feeds are placed downstream of reactor but upstream of mixer; a decanter downstream of mixer with outlets; a decanter downstream of decanter, with outlets; at least a feed downstream of decanter but upstream or directly attached to decanter; and a heating device in fluid connection with outlet, wherein the heating device has a gas outlet in fluid connection with an inlet of decanter, and wherein the heating device has a liquid outlet in fluid connection with a decanter, wherein the decanter is in fluid connection with outlet and an outlet wherein outlet is in fluid connection with a pump, and wherein the pump is in fluid connection with the feed.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A process for purifying linear a-olefins, comprising:
providing an organic phase comprising a linear a-olefin;
providing an alkaline aqueous phase I;
providing an amine;
mixing the linear a-olefin, alkaline aqueous phase I, and amine;
separating the aqueous phase I from the organic phase;
adding water to the organic phase thereby forming an aqueous phase II;
bringing into contact the organic phase with carbon dioxide;
separating the aqueous phase II which comprises a solid formed in the organic phase when contacted with carbon dioxide;
wherein the purified linear a-olefins are obtained in the organic phase.

2. The process of claim 1, wherein at least the bringing into contact the organic phase with carbon dioxide is performed at a pressure in the range of more than 2.0 MegaPascals.

3. The process of claim 1, wherein the process is performed as a continuous process.

4. The process of claim 1, wherein the amine is an organic amine selected from a primary, secondary, tertiary or cyclic amine.

5. The process of claim 4, wherein the organic amine is selected from t-butyl amine, triethyl amine, cyclopentyl amine, t-octyl amine, n-heptyl amine, 2-heptyl amine, hexyl amine, 2-ethylhexyl amine, dihexyl amine, 1,6-diamino hexane, tributyl amine, 1,8-diamino octane, n-dodecyl amine, 3-ethyIheptyl amine and tris-2-ethyl hexyl amine.

6. The process of claim 1, wherein the organic phase comprises at least one component which is non-polar.

7. The process of claim 6, wherein the organic phases further comprises at least one organic solvent selected from aromatic and aliphatic solvents.

8. The process of claim 7, wherein the organic solvent is toluene or cyclohexane.

9. The process of claim 1, wherein the linear a-olefin is obtained by oligomerizing ethylene.

10. The process of claim 1, further comprising a process for purifying amines and carbon dioxide from amine-carbon dioxide complexes, comprising:
providing an aqueous phase II comprising at least one complex of an amine and carbon dioxide, and a gas phase;
heating the aqueous phase II;
disintegrating the complex into at least an amine and carbon dioxide, wherein the carbon dioxide passes from the aqueous phase II into gas phase;
wherein an aqueous phase II′ is formed comprising more amine than carbon dioxide; and
wherein the gas phase comprises more carbon dioxide than amine.

11. The process of claim 10, wherein at least the heating of the aqueous phase II is performed at a pressure in of more than 2.0 MegaPascals.

12. The process of claim 10, wherein the heating of the aqueous phase II is performed until the temperature of the aqueous phase II is 80-100° C.

13. The process of claim 10, wherein at least some water is removed from the aqueous phase II′, wherein an amine enriched aqueous phase II″ is obtained.

14. The process of claim 10, wherein the process is a continuous process.

15. A process for preparing linear a-olefins, comprising:
providing an organic phase comprising at least ethylene and a catalyst;
oligomerizing the ethylene wherein an organic phase comprising a linear a-olefin is obtained forming a compound (a);
providing an alkaline aqueous phase I forming a compound (b);
providing an amine forming a compound (c);
mixing the compounds (a), (b), and (c);
separating the aqueous phase I from the organic phase;
adding water to the organic phase thereby forming an aqueous phase II;
bringing into contact the organic phase with carbon dioxide at a pressure of 2.0 to 3.0 MegaPascals;
separating the aqueous phase II which comprises a solid formed in the organic phase when brought into contact with carbon dioxide at a pressure of 2.0 to 3.0 MegaPascals;
wherein the purified linear a-olefins are obtained in the organic phase;
heating the aqueous phase II to 80-100° C. at a pressure of 2.0 to 3.0 MegaPascals;
disintegrating the complex into at least an amine and carbon dioxide, wherein the carbon dioxide passes from the aqueous phase II into gas phase;
wherein the aqueous phase II contains more amine than carbon dioxide, and
wherein the gas phase comprises more carbon dioxide than amine.

16. The process of claim 15, further comprising removing at least some water from the aqueous phase II, wherein an amine enriched phase II is obtained.

17. The process of claim 15, wherein the process is a continuous process.

18. An apparatus, comprising:
a reactor having at least one feed and at least one outlet, wherein the product outlet is in serial fluid connection with a mixer, wherein at least two feeds are placed downstream of reactor but upstream of mixer;
a decanter downstream of mixer with outlets;
a decanter downstream of decanter, with outlets;
at least a feed downstream of decanter but upstream or directly attached to decanter; and
a heating device in fluid connection with outlet, wherein the heating device has a gas outlet in fluid connection with an inlet of decanter, and wherein the heating device has a liquid outlet in fluid connection with a decanter, wherein the decanter is in fluid connection with outlet and an outlet wherein outlet is in fluid connection with a pump, and wherein the pump is in fluid connection with the feed.
